(19) United States
(12) Patent Application Publication
(10) Pub. No.: US 2011/0200686 A1
(43) Pub. Date: Aug. 18, 2011

(54) COMPOSITIONS OF MODULATORS OF THE WNT/B-CATENIN PATHWAY AND AN N-CINNAMYL-N'BENZHYDRYL PIPERAZINE AND THEIR USE IN TREATING NEOPLASTIC CONDITIONS INCLUDING MALIGNANT MELANOMA

(75) Inventors: Randall T. Moon, Kenmore, WA (US); Travis L. Biechele, Seattle, WA (US); Nathan D. Camp, Seattle, WA (US); Stephen Haggarty, Dorchester, MA (US); Daniel Fass, Cambridge, MA (US); Andy J. Chien, Bellevue, WA (US)

(73) Assignees: UNIVERSITY OG WASHINGTON, Seattle, WA (US); MASSACHUSETTS INSTITUTE OF TECHNOLOGY, Cambridge, MA (US)

(21) Appl. No.: 12/936,695

(22) PCT Filed: Apr. 7, 2009
(86) PCT No.: PCT/US2009/039794

§ 371 Date: May 2, 2011

Related U.S. Application Data
(60) Provisional application No. 61/042,970, filed on Apr. 7, 2008.

(51) Int. Cl. A61K 33/14; A61K 31/4965; A61P 35/00

(52) U.S. Cl. 424/677; 514/255.04

(57) ABSTRACT

The present invention is directed to a composition comprising a modulator of the Wnt/β-catenin pathway or a pharmaceutically acceptable salt thereof and an N-cinnamyl-N′-benzhydryl piperazine or a pharmaceutically acceptable salt thereof. The present invention is also directed to a method of treating a neoplastic condition in a subject by administering the modulator of the Wnt/βcatenin pathway or a pharmaceutically acceptable salt thereof and an N-cinnamyl-N′-benzhydryl piperazine or a pharmaceutically acceptable salt thereof to the subject under conditions effective to treat a neoplastic disorder.

This application claims the benefit of U.S. Provisional Patent Application Ser. No. 61/042,970, filed Apr. 7, 2008, which is hereby incorporated by reference in its entirety.

### FIELD OF THE INVENTION

This invention relates to compositions of modulators of the Wnt/β-catenin pathway and an N-cinnamyl-N′-benzhydryl piperazine and their use in treating neoplastic conditions, including melanoma.

### BACKGROUND OF THE INVENTION

Malignant melanoma accounts for less than five percent of all skin cancers, yet is responsible for 80% of deaths from skin cancer (Tsao et al., N. Engl. J. Med., 351:998-1012 (2004)). The outlook for patients with metastatic melanoma remains quite bleak, with a dismal five-year survival rate of 5-15% that has not changed significantly over the past few decades despite intensive research directed towards developing an effective therapy. The molecular mechanisms underlying melanoma development and progression to metastasis remain unresolved, although recent studies utilizing technologies such as microarray transcriptional profiling have uncovered changes in several different signal transduction pathways in melanoma biology, including the Wnt signaling pathways (Weeraratna A. T., Cancer Metastasis Rev., 24:237-250 (2005)).

Wnt genes encode a family of 19 secreted glycoproteins that activate cellular signaling pathways to control cell fate and differentiation, cell proliferation, and cell motility (Chien et al., Front Biosci., 12:448-457 (2007)). Perturbations of Wnt signaling have subsequently been identified in many different tumor types, including malignant melanoma. Upon binding of a Wnt protein to its cell surface receptor, at least two distinct signaling cascades can be triggered (Chien et al., Front Biosci., 12:448-457 (2007)). The best characterized of these pathways is the canonical Wnt/β-catenin pathway, which is activated upon binding of certain Wnt isoforms to Frizzled/LRP co-receptors triggers downstream events that inhibit the degradation of cytosolic β-catenin. Accumulated cytosolic β-catenin subsequently translocates to the nucleus to control cell fate, cell differentiation, and cell proliferation through the regulation of β-catenin-dependent target genes. Histologically, the visualization of nuclear β-catenin serves as a surrogate measure of Wnt/β-catenin pathway activation in different cancers, including melanoma (Bachmann et al., Clin. Cancer Res., 11:8606-8614 (2005); Kageshita et al., Br. J. Dermatol., 145:210-216 (2001); and Maelandsm et al., Clin. Cancer Res., 9:3383-3388 (2003)).

While Wnt/β-catenin appears to be oncogenic in several cancer models, the exact role of Wnt/β-catenin signaling in melanoma progression remains unresolved (Weeraratna A. T., Cancer Metastasis Rev., 24:237-250 (2005); Moon et al., Nat. Rev. Genet., 5:691-701 (2004); and Morin et al., Science, 275:1787-1790 (1997)). Initial observations that β-catenin staining is more prevalent in benign lesions and early melanomas compared to metastatic lesions led to the hypothesis that Wnt/β-catenin signaling was likely involved in the early stages of melanoma progression. The presence of nuclear β-catenin does not appear to be due to a high frequency of activating mutations of β-catenin or due to inactivating mutations of adenomatous polyposis coli (APC), the tumor suppressor protein frequently dysregulated in colon carcinoma (Omholt et al., Int. J. Cancer, 92:839-842 (2001); Pollock et al., Melanoma Res., 12:183-186 (2002); Reifenberger et al., Int. J. Cancer, 100:549-556 (2002); Rubinfeld et al., Science, 275:1790-1792 (1997); and Worm et al., Oncogene, 23:5215-5226 (2004)). Because the frequency of nuclear β-catenin or APC, the presence of nuclear β-catenin could result from activation of the signaling pathway by secreted Wnt proteins or from as yet unidentified mutations of other Wnt signaling components.

Several studies correlating β-catenin staining to clinical outcomes with tumor microarrays found that the absence of β-catenin (both nuclear and cytoplasmic) in melanomas was associated with a poorer prognosis, suggesting that tumor progression is associated with the absence of Wnt/β-catenin signaling (Bachmann et al., Clin. Cancer Res., 11:8606-8614 (2005); Kageshita et al., Br. J. Dermatol., 145:210-216 (2001); and Maelandsmo et al., Clin. Cancer Res., 9:3383-3388 (2003)). Furthermore, transcriptional profiling of melanoma cell lines has suggested that Wnt/β-catenin signaling regulates a transcriptional signature predictive of less aggressive melanomas (see Hoek et al., Pigment Cell Res., 19:290-302 (2006)).

The present invention is directed to overcoming these and other deficiencies in the art.

### SUMMARY OF THE INVENTION

One aspect of the present invention is directed to a composition comprising a modulator of the Wnt/β-catenin pathway or a pharmaceutically acceptable salt thereof and an N-cinnamyl-N′-benzhydryl piperazine or a pharmaceutically acceptable salt thereof.

Another aspect of the present invention is directed to a method of treating a neoplastic condition in a subject. The method includes administering a modulator of the Wnt/β-catenin pathway or a pharmaceutically acceptable salt thereof and an N-cinnamyl-N′-benzhydryl piperazine or a pharmaceutically acceptable salt thereof under conditions effective to treat a neoplastic disorder.

To date, no studies have used an in vivo model to directly test the rote of Wnt/β-catenin signaling in melanoma progression. To address this issue, a B16 murine melanoma model was used to test the effects of Wnt/β-catenin pathway activation on in vivo tumor growth and metastases. In addition, cultured cells were used to support the hypothesis that Wnt/β-catenin signaling negatively regulates melanoma progression. Cells overexpressing Wnt-3a also exhibited decreased metastasis in viva. This was supported by decreased motility on scratch assays in vitro. Lithium, an approved drug with a long history of clinical usage, is demonstrated here to provide a potential avenue for exploring adjunct therapy in at least a subset of melanoma patients.

### BRIEF DESCRIPTION OF THE DRAWINGS

FIGS. 1A-D are graphs showing that activation of Wnt signaling inhibits the growth of melanoma tumors and the proliferation of melanoma cells. FIG. 1A plots tumor size of B16 tumor cells expressing either GFP or Wnt-3a implanted into the footpads of C57BL6 mice over a two week period. B16 expressing GFP tumors exhibited significantly greater tumor volume than B16 expressing Wnt-3a tumors (p=0.0004 by two-tailed T-test). Each point on the graph represents the average size of four tumors from four different mice for each cell line. Data is representative of three independent experiments (24 mice total). FIGS. 1B-D are graphs indicating the decrease in proliferation of melanoma tumor cells in vitro upon expression of Wnt-3a compared to cells expressing GFP.

FIGS. 2A-B show Wnt3a mediated inhibition of tumor cell metastasis and cell migration. FIG. 2A is a graph showing luciferase activity (representing number of cells) in sentinel lymph node beds in mice having in vivo foot-pad explants of B16 cells expressing luciferase and GFP (B16:GFP) or Wnt-3a (B16:Wnt3a). The decrease in luciferase activity in the sentinel lymph node beds observed in mice having increased Wnt3a expression indicates a decrease in tumor cell metastasis. Bars represent average luciferase activity of four sentinel lymph nodes from four different mice for each cell line. Data is representative of three independent experiments (24 mice total). FIG. 2B shows the results of an in vitro scratch assay showing that cell migration is inhibited in cells expressing Wnt3a (B16:Wnt3a) compared to cells expressing GFP (B16:GFP).

FIGS. 3A-C show in vitro effects of lithium chloride (LiCl) treatment in various melanoma cells. FIG. 3A are images demonstrating nuclear localization of β-catenin upon activation of Wnt signaling by LiCl treatment. FIG. 3B is a dose-response graph showing the effect of LiCl treatment on B16 melanoma cell growth. Sodium chloride (NaCl), which exhibited no effect on cell growth at 20 mM, was used as a negative control. FIG. 3C is a graph comparing the effects of LiCl on cell proliferation in six different human melanoma cell lines. Sodium chloride treatment was used as a control.

FIG. 4 is a graph measuring Wnt pathway activation in a Wnt-responsive luciferase reporter cell line. Wnt activation was observed with LiCl treatment. Combined LiCl (20 mM) and flunarizine(μM) treatment had a synergistic effect on Wnt activation.

FIG. 5 is a graph showing that proliferation of human melanoma cells, measured by Renilla luciferase activity, is inhibited by lithium and by flunarizine (1 μM). LiCl, at 20 mM, acts synergistically with flunarizine to inhibit melanoma cell proliferation (far right bar). Note that proliferation suppression correlates with Wnt signaling, and occurs only at a dose of 20 mM LiCl.

FIG. 6 is a graph showing that in human melanoma cells, flunarizine (1 μM) also synergizes with Wnt-3a conditioned media to activate a Wnt-responsive reporter. QS11, a purine derivative that is known to act synergistically with Wnt-3a to activate the Wnt pathway was used as a positive control (Zhang et al., “Small-Molecule Synergist of the Wnt/beta-Catenin Signaling Pathway,” Proc Natl Acad Sci USA., 104(18):7444-8 (2007), which is hereby incorporated by reference in its entirety).

FIG. 7 is a schematic illustration of a model for treating melanoma as a stem cell tumor.

### DETAILED DESCRIPTION OF THE INVENTION

One aspect of the present invention is directed to a composition comprising a modulator of the Wnt/β-catenin pathway or a pharmaceutically acceptable salt thereof and an N-cinnamyl-N′-benzhydryl piperazine or a pharmaceutically acceptable salt thereof.

In accordance with this aspect of the present invention, the N-cinnamyl-N′-benzhydryl piperazine has the formula:

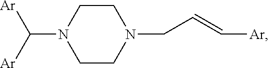

where each Ar is selected from the group consisting of phenyl or fluorophenyl, with at least one Ar being fluorophenyl. In certain embodiments, the N-cinnamyl-N′-benzhydryl piperazine has the formula:

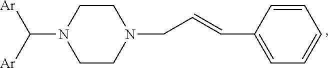

where Ar¹ is fluorophenyl. In a preferred embodiment, the N-cinnamyl-N′-benzhydryl piperazine is flunarizine. Other suitable N-cinnamyl-N′-benzhydryl piperazines include disubstituted piperazines such as those described in U.S. Pat. No. 3,773,939 to Janssen, which is hereby incorporated by reference in its entirety, or derivatives thereof.

Flunarizine (1-[bis(4-fluorophenyl)methyl]-4-cinnamyl-piperazine) is a drug classified as a calcium channel blocker. Flunarizine is a selective calcium entry blocker with calmodulin binding properties and histamine H1 blocking activity.

In accordance with this aspect of the invention suitable modulators of the Wnt/β-catenin pathway include lithium or a pharmaceutically acceptable salt thereof, or an inhibitor of a GSK-3 kinase or a pharmaceutically acceptable salt thereof. In a preferred embodiment the GSK-3 kinase is GSK3β.

The lithium salt can be selected from the group consisting of lithium chloride, lithium citrate, lithium carbonate, lithium orotate, and mixtures thereof. In a preferred embodiment, the lithium salt is lithium chloride.

In addition to lithium, other modulators of the Wnt/β-catenin pathway suitable for inclusion in the composition of the present invention include β-catenin signal-promoting agents. As used herein, “β-catenin signal-promoting agents” refer to agonists or antagonists of positive or negative signaling molecules, respectively, of β-catenin signaling, i.e., any agent that activates β-catenin signaling through inhibition of GSK-3 in the presence or absence of Wnt signaling. For example, activation of β-catenin signaling in the absence of Wnt signaling can occur by activation of integrin linked kinase, activation of p53 leading to activation of Siah1, or activation of FGF signaling. β-catenin signal-promoting agents further include any signaling molecule that activates β-catenin target genes via inhibition of GSK-3 having a therapeutic potential. β-catenin signal-promoting agents also refer to any signaling molecule that activates β-catenin target genes independent of GSK-3 having therapeutic potential. Activation of β-catenin target genes without inhibiting GSK-3 can be achieved by inhibition (e.g., by drug therapy, RNAi therapy or gene therapy) of any inhibitor of β-catenin function, including, but not limited to, APC, Axin, Chibby, ICAT, Groucho, and CtBP.

Other modulators of the Wnt/β-catenin pathway include Wnt signal- or β-catenin signal-promoting agent. As used herein, a “Wnt signal- or β-catenin signal-promoting agent” refers to one or more of the following: a nucleic acid molecule comprising a nucleotide sequence that encodes a Wnt polypeptide; a polypeptide comprising an amino acid sequence of a Wnt polypeptide; a nucleic acid molecule comprising a nucleotide sequence that encodes an activated Wnt receptor; a polypeptide comprising an amino acid sequence of an activated Wnt receptor; a small organic molecule that promotes Wnt/β-catenin signaling, Notch signaling, or Hedgehog signaling; a small organic molecule that inhibits the expression or activity of a Wnt, β-catenin, Notch, or Hedgehog antagonist; an antisense oligonucleotide that inhibits expression of a Wnt, β-catenin, Notch, or Hedgehog antagonist; a ribozyme that inhibits expression of a Wnt, β-catenin, Notch, or Hedgehog antagonist; an RNAi construct, siRNA, or shRNA that inhibits expression of a Wnt, β-catenin, Notch, or Hedgehog antagonist; an antibody that binds to and inhibits the activity of a Wnt, β-catenin, Notch, or Hedgehog antagonist; a nucleic acid molecule comprising a nucleotide sequence that encodes a β-catenin polypeptide; a polypeptide comprising an amino acid sequence of a β-catenin polypeptide; a nucleic acid molecule comprising a nucleotide sequence that encodes a Lef-1 polypeptide; and a polypeptide comprising an amino acid sequence of a Lef-1 polypeptide.

Other suitable modulators of the Wnt/β-catenin pathway include agents that inhibit GSK3. Exemplary GSK3 inhibitors include, but are not limited to, a nucleic acid molecule comprising a nucleotide sequence that encodes a dominant negative GSK-3, GSK3α, or GSK3β polypeptide; a polypeptide comprising an amino acid sequence of a dominant negative GSK-3, GSK3α, or GSK3β polypeptide; a small organic molecule that binds to and inhibits the expression or activity of GSK-3, GSK3α, or GSK3β; an RNAi construct, siRNA, or shRNA that binds to and inhibits the expression and/or activity of GSK-3, GSK3α, or GSK3β; an antisense oligonucleotide that binds to and inhibits the expression and/or activity of GSK-3, GSK3α, or GSK3β; an antibody that binds to and inhibits the expression and/or activity of GSK-3, GSK3α, or GSK3β; a ribozyme that binds to and inhibits the expression of GSK-3, GSK3α, or GSK3β; and any GSK-3 independent reagent that activates β-catenin target genes similar in effect to GSK-3 inhibition.

Exemplary Wnt/β-catenin signal-, Notch signal- or Hedgehog signal-promoting agents include, but are not limited to, lithium chloride or other GSK-3 inhibitors, as exemplified in U.S. Pat. Nos. 6,057,117 to Harrison et al., and 6,608,063 to Nuss et al., and U.S. Patent Publication Nos. 20040092535 to Barsanti et al., and 20040209878 to Guzi et al., which are hereby incorporated by reference in their entirety. Other suitable agents include the ATP-competitive, selective GSK-3 inhibitors CHIR-911 and CHIR-837 (also referred to as CT-99021 and CT-98023 respectively) developed by Chiron Corporation (Emergyville, Calif.). These inhibitors were purified >95% by high-performance liquid chromatography. CHIR-911 was formulated in 10% captisol solution for administration in vivo by intraperitoneal injection, with a half-maximal effective concentration [EC₅₀] of 766 nM and >10,000 fold selectivity for GSK-3 (Ring et al., Diabetes 52:588-595 (2003), which is hereby incorporated by reference in its entirety). CHIR-837 was formulated in DMSO for in vitro use, with an EC₅₀ of 375 μM and >5,000 fold selectivity for GSK-3 (Cline et al., Diabetes 51:2903-2910 (2002), which is hereby incorporated by reference in its entirety). The GSK-3 inhibitor CHIR025 is also suitable for use in accordance with this aspect of the invention (Kelley S., Exp. Neurol., 188:378-386 (2004), which is hereby incorporated by reference in its entirety).

Other exemplary Wnt/β-catenin signal-promoting agents include, but are not limited to, GSK-3 inhibitors such as SB-216763 and SB-415286, developed by Glaxo Smith Kline (Eldar-Finkelman et al., TRENDS in Molecular Medicine 8(3):126-132 (2002), and Patel et al., Biochem. Soc. Trans. 32(5):803-808 (2004), which are hereby incorporated by reference in their entirety). Calbiochem® Alzheimer's and Other Neurodegenerative Disease Research Tools, EMD Biosciences, Inc. San Diego, Calif., pgs 1-32 (2003), which is hereby incorporated by reference in its entirety, additionally discloses aloisine A, Aloisine RP106, alsterpaullone, a thiadiazolidinone analog, a 2-thio[1,3,4]-oxadiazole-pyridyl derivative, an oxothiadiazolidine-3-thione analog, indirubin-3′-monoxime, 5-iodo-indirubin-3′-monoxime, indirubin-3′-monoxime-5-sulphonic acid, and kanpaullone as small molecule GSK-3 inhibitors and GSK-3 peptide inhibitors H-KEAPPAPPQSpP-NH₂ (SEQ ID NO:1) and Myr-N-GKEAPPAPPQSpP-NH₂(SEQ ID NO:2). Still further exemplary GSK-3 inhibitors include, but are not limited to, compound 603281-31-8 developed by Lilly (Kukarni et al., J. Bone Miner. Res., 21:910-920 (2006), which is hereby incorporated by reference in its entirety), the FRATtide peptide (Bax et al., Structure, 9:1143-1152 (2001), which is hereby incorporated by reference in its entirety), 6-bromoindirubin-3′ oxime (Parkitna et al., JPET 319(2):832-9 (2006), which is hereby incorporated by reference in its entirety), and retinoic acid (Eisinger et al., J. Biol. Chem., 282(40):29394-29400 (2007), which is hereby incorporated by reference in its entirety).

Other suitable GSK-3 inhibitors include zinc, Akt, PKC, PKA, p90RSK, thymoleptics (e.g. valproate, MAOIs, fluoxetine, imipramine, clozapine, risperidone, and haloperidol), estrogen, L803-mts, and AR-A014418 (Gould et al., Neuropsychopharm, 30(7):1223-37 (2005), which is hereby incorporated by reference in its entirety. Stimulators of 5-HT_{1A} receptors that inhibit GSK-3 are described in Beaulieu J., Int'l J. Neuropsychopharm., 1-4 (2006), which is hereby incorporated by reference in its entirety.

In certain embodiments, the composition of the present invention also includes a pharmaceutically acceptable excipient or carrier.

Another aspect of the present invention is directed to a method of treating a neoplastic condition in a subject. The method includes administering a modulator of the Wnt/β-catenin pathway or a pharmaceutically acceptable salt thereof and an N-cinnamyl-N′-benzhydryl piperazine or a pharmaceutically acceptable salt thereof under conditions effective to treat a neoplastic disorder.

The modulator of the Wnt/β-catenin pathway or a pharmaceutically acceptable salt thereof and an N-cinnamyl-N′-benzhydryl piperazine or a pharmaceutically acceptable salt thereof may include embodiments substantially the same as those described above. In a preferred embodiment, the modulator of the Wnt/β-catenin pathway is lithium chloride and the N-cinnamyl-N′-benzhydryl piperazine is flunarizine.

The method of the present invention may include selecting a subject with a neoplastic condition and administering the modulator of the Wnt/β-catenin pathway or a pharmaceutically acceptable salt thereof and an N-cinnamyl-N′-benzhydryl piperazine or a pharmaceutically acceptable salt thereof to the selected subject. The neoplastic condition may be a non-malignant or malignant condition. Malignant neoplasms that may be treated in accordance with this aspect of the invention include, but are not limited to carcinomas (e.g., adenocarcinomas or squamous cell carcinoma), Hodgkin's and non-Hodgkin's lymphomas, leukemias, melanomas, and sarcomas. In a preferred embodiment, the neoplastic condition is melanoma and the subject is human.

In a preferred embodiment, the administering is carried out by treating the subject with a composition comprising the modulator of the Wnt/β-catenin pathway or a pharmaceutically acceptable salt thereof and the N-cinnamyl-N′-benzhydryl piperazine or a pharmaceutically acceptable salt thereof.

The compositions of the present invention can be administered orally, parenterally, for example, subcutaneously, intravenously, intramuscularly, intraperitoneally, by intranasal instillation, by inhalation, or by application to mucous membranes, such as, that of the nose, throat, and bronchial tubes. They may be administered alone or with suitable pharmaceutical carriers, and can be in solid or liquid form such as, tablets, capsules, powders, solutions, suspensions, or emulsions.

The active compounds of the present invention may be orally administered, for example, with an inert diluent, or with an assimilable edible carrier, or they may be enclosed in hard or soft shell capsules, or they may be compressed into tablets, or they may be incorporated directly with the food of the diet. For oral therapeutic administration, these active compounds may be incorporated with excipients and used in the form of tablets, capsules, elixirs, suspensions, syrups, and the like. Such compositions and preparations should contain at least 0.1% of active compound. The percentage of the compound in these compositions may, of course, be varied and may conveniently be between about 2% to about 60% of the weight of the unit. The amount of active compound in such therapeutically useful compositions is such that a suitable dosage will be obtained. Preferred compositions according to the present invention are prepared so that an oral dosage unit contains between about 1 and 250 mg of active compound.

The tablets, capsules, and the like may also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it may contain, in addition to materials of the above type, a liquid carrier, such as a fatty oil.

Various other materials may be present as coatings or to modify the physical form of the dosage unit. For instance, tablets may be coated with shellac, sugar, or both. A syrup may contain, in addition to active ingredient, sucrose as a sweetening agent, methyl and propylparabens as preservatives, a dye, and flavoring such as cherry or orange flavor.

These active compounds may also be administered parenterally. Solutions or suspensions of these active compounds can be prepared in water suitably mixed with a surfactant, such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

The compounds of the present invention may also be administered directly to the airways in the form of an aerosol. For use as aerosols, the compounds of the present invention in solution or suspension may be packaged in a pressurized aerosol container together with suitable propellants, for example, hydrocarbon propellants like propane, butane, or isobutane with conventional adjuvants. The materials of the present invention also may be administered in a non-pressurized form such as in a nebulizer or atomizer.

The compounds of the present invention may also be administered directly to the airways in the form of a dry powder. For use as a dry powder, the compounds of the present invention may be administered by use of an inhaler. Exemplary inhalers include metered dose inhalers and dry powdered inhalers. A metered dose inhaler or “MDI” is a pressure resistant canister or container filled with a product such as a pharmaceutical composition dissolved in a liquefied propellant or micronized particles suspended in a liquefied propellant. The correct dosage of the composition is delivered to the patient. A dry powder inhaler is a system operable with a source of pressurized air to produce dry powder particles of a pharmaceutical composition that is compacted into a very small volume. For inhalation, the system has a plurality of chambers or blisters each containing a single dose of the pharmaceutical composition and a select element for releasing a single dose.

Suitable powder compositions include, by way of illustration, powdered preparations of the active ingredients thoroughly intermixed with lactose or other inert powders acceptable for intrabronchial administration. The powder compositions can be administered via an aerosol dispenser or encased in a breakable capsule which may be inserted by the patient into a device that punctures the capsule and blows the powder out in a steady stream suitable for inhalation. The compositions can include propellants, surfactants, and co-solvents and may be filled into conventional aerosol containers that are closed by a suitable metering valve.

Aspects of the present invention include new combination drug therapies for treatment of malignant melanoma. Lithium is currently used as an anti-manic agent for patients with bipolar disorder. One of the biological effects of lithium is inhibition of the kinase GSK-3β. Inhibition of GSK-3β activates the Wnt/β-catenin-TCF/LEF transcriptional pathway. Studies on melanoma suggest that activation of Wnt/β-catenin signaling negatively regulates melanoma progression, and is associated with improved patient prognosis. See Bachmann et al., Clin Cancer Res, 8606-8614 (2005) and Kageshita et al., Br J. Dermatol, 145: 210-216 (2001), which are hereby incorporated by reference in their entirety. Activation of the Wnt/β-catenin pathway in melanoma cells can inhibit growth and metastasis in murine models and inhibit growth of human melanoma cell lines. In addition, there is evidence that activation of the Wnt/β-catenin pathway in melanoma cells could also affect their state of differentiation and activate transcriptional profiles seen in less aggressive melanomas.

Systemic treatments for melanoma are currently limited, particularly in the case of metastatic disease. The use of drugs that could inhibit melanoma progression, such as lithium and flunarizine, could augment existing therapies to boost their efficacy. In addition, the observed effects on the differentiation state of melanoma cells caused by activation of the Wnt/β-catenin pathway may also render melanoma tumors less aggressive, again allowing for increased efficacy of other existing therapies. It might be possible to use lower doses of the components in the present combination therapy, reducing the risk of toxic side effects.

### EXAMPLES

### Example 1

### Cell Lines

B 16 murine melanoma cells expressing firefly luciferase have been previously described and were used as the parental line for experiments described herein. Human melanoma UACC 1273 and M92047 (National Institute of Aging; Baltimore, Md.) and human melanoma cell lines Mel375, A2058, Mel 29.6 and Mel501 (Fred Hutchinson Cancer Research Institute; Seattle, Wash.) were also used. Cell lines overexpressing green fluorescent protein (GFP) or human Wnt isoforms were generated by lentiviral transduction. Sequences for different Wnt isoforms were amplified by polymerase chain reaction (PCR) and cloned into third generation lentiviral vectors derived from backbone vectors (Dull et al., J Virol, 72: 8463-71 (1998) which is hereby incorporated by reference in its entirety. These lentiviral vectors contained an EF1-alpha promoter driving a bi-cistronic message encoding a Wnt plus GFP. Cells were sorted by FACS for GFP expression, with the goal of obtaining cells with approximately equivalent levels of GFP expression.

### Example 2

### Cell Culture

B16 murine melanoma cells were cultured in Dulbeccos modified Eagle's media supplemented with 2% Fetal Bovine Serum, and 1% antibiotic/antimycotic (Invitrogen; Grand Island, N.Y.). Human melanoma lines A375, M92047, A2058, MeI 29.6, Mel501, and Mel526 were cultured in DMEM supplemented with 2% FBS and 1% antibiotic/antimycotic. Human melanoma line UACC 1273 was cultured in RPMI (Invitrogen; Grand Island, N.Y.) supplemented with 2% FBS, and 1% antibiotic/antimycotic. All cell lines were cultured in the presence of 0.02% Plasmocin (InvivoGen; San Diego, Calif.).

### Example 3

### Measurement of Wnt Pathway Activation Using a Reporter Assay

Wnt3a conditioned media was collected from sub-confluent melanoma cell lines, and this media was tested for its ability to activate Wnt/β-catenin signaling in UACC 1273 melanoma cells that were stably transduced with a Wnt/β-catenin-responsive firefly luciferase reporter and a constitutive Renilla luciferase gene used for normalization (Cignal TCF/LEF transcriptional reporter construct; SABiosciences, Frederick, Md.). Conditioned media was spun down to clear cell debris and then incubated with reporter cells overnight. Activation of the Wnt/β-catenin reporter was measured using a DLR assay kit (Promega; Madison, Wis.).

### Example 4

### In Vivo Tumor Inoculation and Measurements of Lymph Node Metastasis

For in vivo tumor inoculation, 4×10⁵ B16 cells overexpressing GFP (B16:GFP) or Wnt3a (B16:Wnt3a) were injected in a volume of ˜40 μA PBS into the left rear footpad of C57BL6 mice. Tumor size was evaluated by measuring the maximal depth of the tumor within the footpad (mm) and then subtracting the baseline foot thickness of the contralateral (non-manipulated) footpad of the same animal. Tumor size was then approximated by multiplying tumor depth, maximal tumor width, and maximal perpendicular length (in mm). Following euthanasia on day 14 after tumor inoculation, the draining popliteal lymph node of the injected footpad was removed from a given mouse and homogenized with the back of a syringe in 500 μl Glo lysis buffer (Promega; Madison, Wis.). Subsequently, the lysate was passed through a 40 μm filter and 100-μl aliquots were then assayed in the presence of luciferin (Promega; Madison, Wis.) using a MLX 96-well luminometer (Thermo Labsystems; Helsinki, Finland). The mean value of luciferase activity at each time point was calculated based on triplicate samples. Units are in relative light units (RLU). The different cells lines showed similar levels of luciferase activity at the time of footpad injection.

### Example 5

### Cell Proliferation Assays

For cell counts by hematocytometer, cells were plated at a uniform density (usually between 10,000 to 25,000 cells per well) in a 12 or 24 well tissue culture plate in the appropriate media supplemented with 2% FBS. Cells were then incubated at 37° C. at 5% CO₂ for three to seven days based on individual cell doubling time. At the end of the timecourse, cells were trypsinized, resuspended in the appropriate media, and counted using a standard hematocytometer. Dead cells were identified by 0.4% Trypan Blue stain. Cell proliferation experiments were performed with a minimum of six biologic replicates. Similar results were observed for all cell lines using the MTT assay (ATCC; Mannassas, Va.), performed according to manufacturer's protocol. For relative cell proliferation assays of B16:GFP cells incubated with lithium chloride or sodium chloride, cell proliferation was measured by relative firefly luciferase units (Promega; Madison, Wis.).

### Example 6

### Immunohistochemistry

Cells were grown on 18 mm glass coverslips, and after 48-72 hours, cells were fixed using 4% paraformaldehyde, permeabilized using 0.25% Triton X-100, and then blocked with 10% goat serum. A polyclonal rabbit anti-β-catenin antibody (Sigma, Cat# C2206) was diluted 1:200 in antibody buffer (0.1% Tween and 2% goat serum in PBS). Cells were incubated with the antibody overnight at 4° C. Goat anti-rabbit Alexa Fluor-568 antibody (Molecular Probes; Eugene, Oreg.) was diluted 1:1000 in antibody buffer, filtered, and added to each well. Cells were counterstained for nucleic acid with DAN (Molecular Probes; Eugene, Oreg.). Cells were imaged with oil immersion at 60× objective on a Nikon Upright Eclipse E600 (Nikon, USA).

### Example 7

### Drug Treatment

Flunarizine and QS11 were solubilized in dimethyl-sulfoxide (DMSO) vehicle, which was used as a control (vol/vol) for the described experiments. Drug incubations were 24-48 hours in duration.

### Example 8

### Wnt3a Modulation Negatively Regulates Melanoma Growth

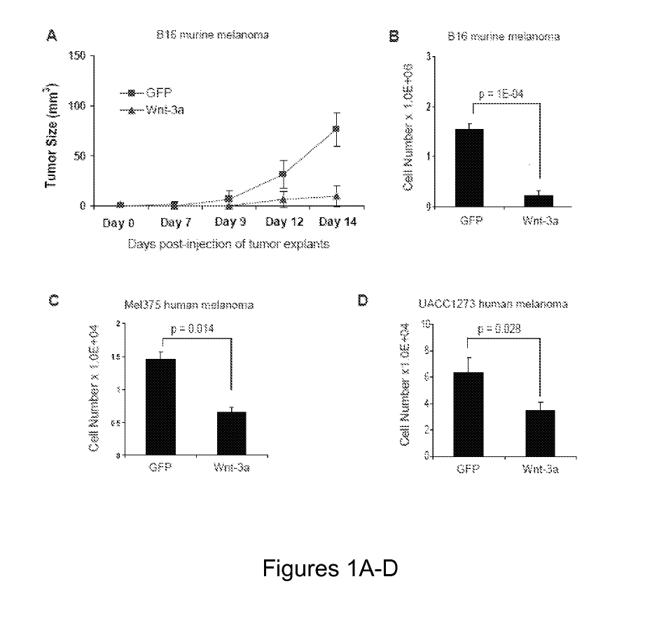

Several lines of evidence are provided to support the hypothesis that activation of Wnt/β-catenin signaling can negatively regulate melanoma growth and progression. First, a decrease in tumor cell proliferation was observed with Wnt3a overexpression in B16 murine melanoma cells (FIG. 1B), Mel375 human melanoma cells (FIG. 1C), and UACC 1273 human melanoma cells (FIG. 1D). Similarly, overexpression of Wnt1, which also activates the Wnt/β-catenin pathway, also decreased tumor cell proliferation.

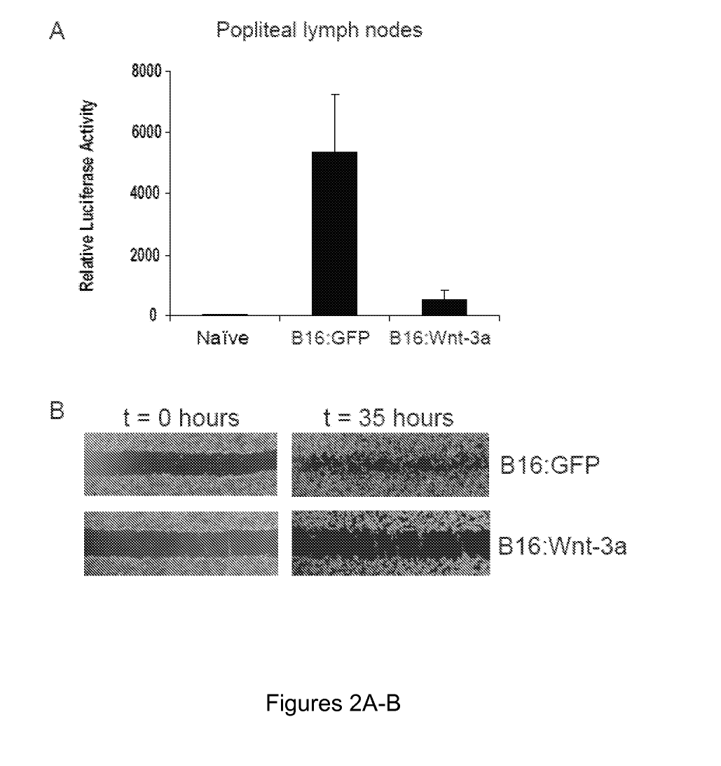

This decrease in tumor cell proliferation upon activation of the Wnt/β-catenin pathway by Wnt3a overexpression was also observed in vivo. Tumor growth of B16 melanoma cells overexpressing Wnt3a was significantly inhibited compared to the tumor growth of B16 cells overexpressing GFP (FIG. 1A). Staining of tumor explants ruled out increased apoptosis as a cause for the decreased tumor size. The lack of B16 tumor cells detected in the sentinel lymph node beds of mice with Wnt3a-expressing tumors confirms that the decreased growth seen in the tumor explants was not due to increased tumor metastasis (FIG. 2A). A scratch assay, used to measure cell migration, confirmed that Wnt3a overexpression in tumor cells inhibited cell migration compared to control cells expressing GFP.

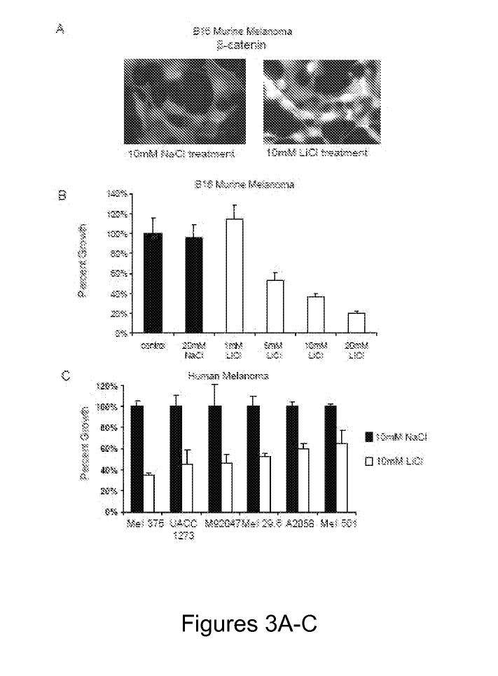

The above experimental results show that Wnt-3a, an activator of the Wnt/β-catenin signaling pathway, can negatively regulate the proliferation of both human and murine melanoma cell lines in vitro, and in an in vivo tumor model. The decrease in tumor cell proliferation observed in cells overexpressing Wnt3a (FIGS. 1B-D) was phenocopied in both human and murine melanoma cell lines with lithium chloride, a well-characterized activator of the Wnt/β-catenin pathway (FIGS. 3B-C). Lithium chloride treatment also caused the nuclear localization of β-catenin in B16 melanoma cells, further confirming the activation of the Wnt/β-catenin pathway in these cells (FIG. 3A). Together, these findings support the hypothesis that activation of Wnt/β-catenin signaling negatively regulates melanoma progression. These findings could explain why nuclear β-catenin staining, a surrogate histologic marker of Wnt/β-catenin signaling activity, appears to be lost during melanoma progression.

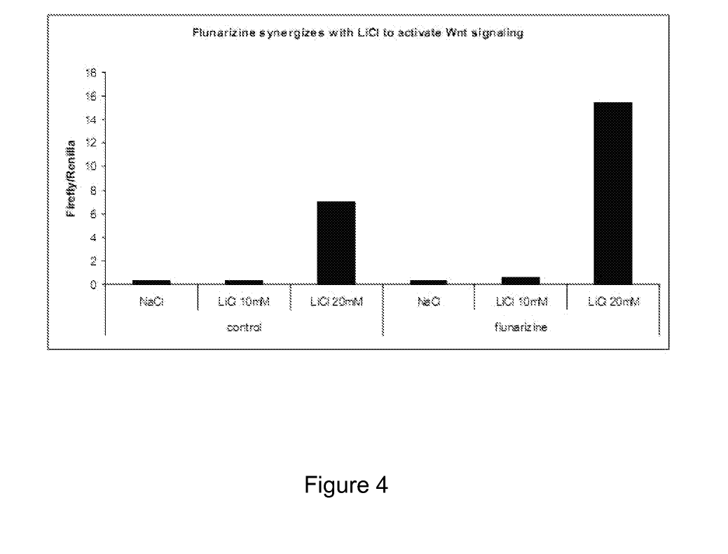

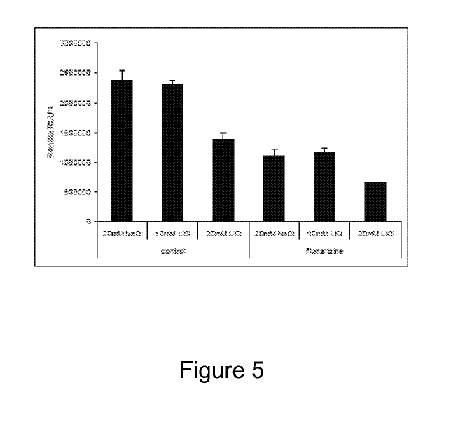

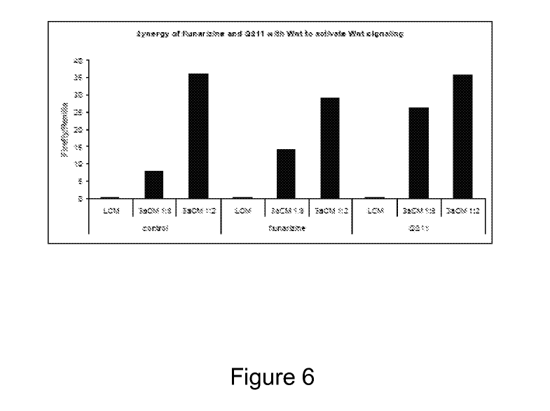

A TCF/LEF transcriptional reporter gene cell line used to monitor the activation of the Wnt signal transduction pathway was utilized in a high throughput screen of a library of chemical compounds to identify compounds that synergize with lithium in activating the Wnt/β-catenin pathway. Flunarizine, a N-cinnamyl-N′-benzhydryl piperazine, enhanced lithium-induced activation of the TCF/LEF reporter gene by ˜100 fold (FIG. 4). Flunarizine also synergistically enhanced the activation of the Wnt signaling pathway by Wnt3a (FIG. 6). The synergistic effect of lithium and flunarizine on the activation of the TCE/LEF reporter in melanoma cells, suggested that a combination treatment consisting of these drugs could present a viable method for inhibiting melanoma cell growth and metastatis in vivo. In support of this hypothesis, a synergistic inhibition of melanoma cell growth and proliferation was observed with the combined treatment of lithium and flunarizine in vitro (FIG. 5).

Flunarizine works with both Wnt-3a and with lithium chloride to increase activation of the Wnt/β-catenin pathway as measured by transcriptional reporter assays (FIGS. 4 and 6). The ability of both lithium and flunarizine to interact with Wnt-3a suggests that these drugs may be able to augment Wnt/β-catenin signaling in tumors with nuclear β-catenin (a surrogate marker of Wnt/β-catenin pathway activation in tumors).

In addition, the combination of flunarizine and lithium chloride decreases melanoma cell proliferation in parallel with its effects on augmenting activation of the Wnt/β-catenin pathway (FIG. 5). The synergistic effects of the combination of drugs potentially allows for lower drug doses, minimizing toxicity.

Wnt3a and Wnt/β-catenin signaling are implicated as major regulators of melanocyte biology during development. In the developing neural crest, activation of Wnt/β-catenin signaling can bias cells towards a melanocytic lineage at the expense of neuronal lineages. Transcriptional profiling suggests that Wnt3a could be affecting melanoma cell fate in a manner similar to the effects of Wnt/β-catenin signaling on neural crest-derived melanocyte precursors.

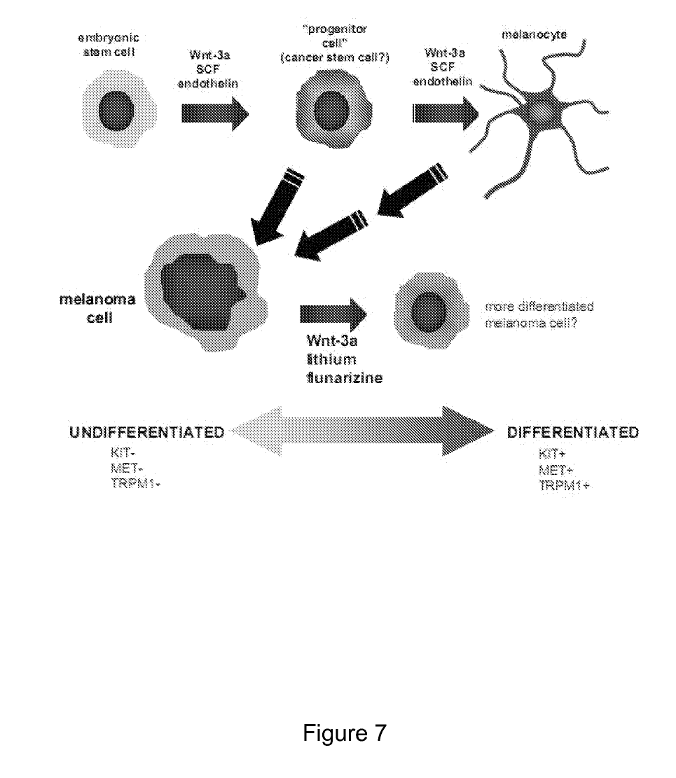

Melanoma has been described as a tumor of possible stem cell origin. FIG. 7 shows the potential relationship between melanocyte differentiation and the progression of melanoma. Only three factors are needed to generate functional melanocytes from human embryonic stem cells: Wnt-3a, stem cell factor (SCF), and endothelin-1. The expression of Wnt-3a in melanoma cells can lead to the expression of markers normally seen in more differentiated cells (KIT, MET and TRPM1). Conceivably, melanomas may result from the de-differentiation of a mature melanocyte or from the aberrant differentiation/de-differentiation of an intermediate progenitor cell involved in melanocyte differentiation.

The findings described herein suggest that one potential strategy for targeting melanoma, as well as other cancers, is to exploit the same signaling pathways involved in regulating cell fate during embryonic development in order to bias cancer cells towards a more differentiated state. Since lithium can mimic the effects of Wnt3a, and funarizine and QS11 synergize the effects of lithium and Wnt3a, drug combinations that activate Wnt/β-catenin signaling could be exploited to bias melanomas towards a more differentiated state and render them less proliferative and less metastatic. Because lithium and flunarizine work synergistically, using them in combination will permit overall lower doses of each individual drug to decrease toxicity profiles. Patients could potentially benefit from the activation of Wnt/β-catenin by small molecules that mimic the effects of Wnt3a, regardless of the β-catenin status of their tumors. Ultimately, these types of differentiation strategies may provide a means to therapeutically address a cancer that currently has very limited options for metastatic disease.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A composition comprising:
a modulator of the Wnt/β-catenin pathway or a pharmaceutically acceptable salt thereof; and
an N-cinnamyl-N′-benzhydryl piperazine or a pharmaceutically acceptable salt thereof.

2. The composition of claim 1 further comprising a pharmaceutically acceptable excipient or carrier.

3. The composition of claim 1, wherein the modulator of the Wnt/β-catenin pathway is a lithium compound or a pharmaceutically acceptable salt thereof.

4. The composition of claim 3, wherein the lithium compound is selected from the group consisting of lithium chloride, lithium citrate, lithium carbonate, lithium orotate, and mixtures thereof.

5. The composition of claim 3, wherein the lithium compound is lithium chloride.

6. The composition of claim 1, wherein the modulator of the Wnt/β-catenin pathway is an inhibitor of a GSK-3.

7. The composition of claim 1, wherein the modulator of the Wnt/β-catenin pathway is an inhibitor of GSK-3β.

8. The composition of claim 1, wherein the N-cinnamyl-N′-benzhydryl piperazine has the formula: 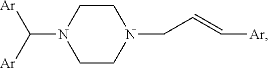
wherein,
Ar is selected from the group consisting of phenyl or fluorophenyl, with at least one Ar being fluorophenyl.

9. The composition of claim 1, wherein the N-cinnamyl-N′-benzhydryl piperazine has the formula: 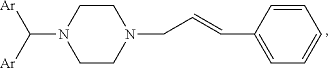
wherein,
Ar¹ is fluorophenyl.

10. The composition of claim 1, wherein the N-cinnamyl-N′-benzhydryl piperazine is flunarizine.

11. A method of treating a neoplastic condition in a subject, said method comprising:
administering a modulator of the Wnt/β-catenin pathway or a pharmaceutically acceptable salt thereof and an N-cinnamyl-N′-benzhydryl piperazine or a pharmaceutically acceptable salt thereof to the subject under conditions effective to treat the neoplastic disorder.

12. The method of claim 11 further comprising:
selecting a subject with a neoplastic condition, wherein the modulator of the Wnt/β-catenin pathway or a pharmaceutically acceptable salt thereof and the N-cinnamyl-N′-benzhydryl piperazine or a pharmaceutically acceptable salt thereof is administered to the selected subject.

13. The method of claim 12, wherein the neoplastic condition is cancer.

14. The method of claim 13, wherein the cancer is melanoma.

15. The method of claim 11, wherein the modulator of the Wnt/β-catenin pathway or a pharmaceutically acceptable salt thereof and the N-cinnamyl-N′-benzhydryl piperazine or a pharmaceutically acceptable salt thereof further comprises a pharmaceutically acceptable excipient or carrier.

16. The method of claim 11, wherein the modulator of the Wnt/β-catenin pathway is a lithium compound selected from the group consisting of lithium chloride, lithium citrate, lithium carbonate, lithium orotate, and mixtures thereof.

17. The method of claim 11, wherein the lithium compound is lithium chloride.

18. The method of claim 11, wherein said administering is carried out orally, intradermally, intramuscularly, intraperitoneally, intravenously, subcutaneously, or intranasally.

19. The method of claim 11, wherein the modulator of the Wnt/β-catenin pathway is an inhibitor of a GSK-3.

20. The method of claim 11, wherein the modulator of the Wnt/β-catenin pathway is an inhibitor of GSK-3β.

21. The method of claim 11, wherein the N-cinnamyl-N′-benzhydryl piperazine has the formula: 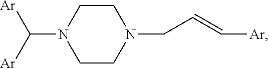
wherein,
Ar is selected from the group consisting of phenyl or fluorophenyl, with at least one Ar being fluorophenyl.

22. The method of claim 11, wherein the N-cinnamyl-N′-benzhydryl piperazine has the formula: 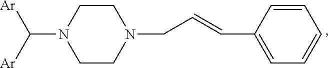
wherein,
Ar¹ is fluorophenyl.

23. The method of claim 11, wherein the N-cinnamyl-N′-benzhydryl piperazine is flunarizine.

24. The method of claim 11, wherein said administering is carried out by treating the subject with a composition comprising the modulator of the Wnt/β-catenin pathway or a pharmaceutically acceptable salt thereof and the N-cinnamyl-N′-benzhydryl piperazine or a pharmaceutically acceptable salt thereof.
